(19) United States
(12) Patent Application Publication
(10) Pub. No.: US 2010/0247380 A1
(43) Pub. Date: Sep. 30, 2010

(54) Module for a Modular Microfluidic System

(76) Inventors: Astrid Lohf, Erlangen (DE); Reinhold Schneeberger, Seukendorf (DE); Robert Sendner, Furth (DE); Johann Sippl, Nurnberg (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/225,838

(22) PCT Filed: Mar. 28, 2007
(86) PCT No.: PCT/EP2007/052954

§ 371 Date: Jun. 18, 2010

(30) Foreign Application Priority Data
Mar. 30, 2006 (DE) 10 2006 014 845.2

(51) Int. Cl. G01N 33/00

(52) U.S. Cl. 422/68.1

(57) ABSTRACT

In order to be able to introduce fluid components extraneous to the system into a modular microfluidic system comprising modules arranged alongside one another which can be connected to one another with fluid connection by connecting parts comprising connecting channels with short connecting paths and good temperature controllability, a module with the following features is provided: the module has a plate-shaped microfluidic part which comprises a fluid channel system and on whose top side, in edge regions to the potentially adjacent modules of the microfluidic system has fluid connections, the fluidic connection to adjacent modules being establishable by means of the connecting parts adjacent in edge regions on the upper side, below the microfluidic part is arranged an insulation vessel which can be filled and flowed through by a temperature control fluid and is concluded at the to by the microfluidic part which serves as a lid.

### CROSS REFERENCE TO RELATED APPLICATIONS

This application is the US National Stage of International Application No. PCT/EP2007/052954, filed Mar. 28, 2007 and claims the benefit thereof. The International application claims the benefits of German application No. 10 2006 014 845.2 filed Mar. 30, 2006, both of the applications are incorporated by reference herein in their entirety.

### FIELD OF INVENTION

The invention relates to a module for a modular microfluidic system in which modules arranged adjacent to one another in a row can be fluidically connected to one another by means of connecting parts containing connection channels.

### BACKGROUND OF THE INVENTION

Modular microfluidic systems, as similarly known from WO 01/36085 A1, WO 01/73823 A2, WO 02/065221 A2 and WO 2005/107937 A1, consist of a plurality of modules which each contain a microfluidic part and an associated electrical control unit and can be mounted by their rear sides adjacent to one another in series on a mounting rail. The control units of the different modules are interconnected via an electrical line bus and the microfluidic parts via a fluid bus. As WO 02/065221 A2 shows, the fluid bus can be formed by connecting the microfluidic parts of respectively adjacent modules to one another via connecting parts containing connection channels and spanning the respective modules.

In the individual microfluidic parts, module-specific functions are carried out in the context of the fluid treatment in the microfluidic system, treatment of fluids being understood to mean in particular their analysis and/or synthesis including the auxiliary functions necessary therefor, such as e.g. pumping, temperature control, filtering, etc.; the fluids can be liquids, gases or solids conveyed by carrier fluids. Other micro- or macrofluidic units such as pumps, mass flow meters etc. which cannot readily be implemented in the microfluidic parts can be connected to the microfluidic parts, such as e.g. microreactors, mixers, dwell tanks, etc.

Moreover there is also the requirement to enable system-external fluidic components such as e.g. system-external (micro)reactors, mixers, dwell tanks, preheaters, etc. to be integrated into existing microfluidic systems. For that purpose external fluid terminals can be provided for example at the connecting parts between the modules for the purpose of connecting the system-external fluidic components via tubes or capillaries. In this way fluids are channeled out of the microfluidic system into the system-external fluidic component and thence routed back again into the microfluidic system. For controlling the temperature of system-external fluidic components of this kind, the user is reliant on conventional thermostats, while the connecting lines between the fluidic component and the thermostat are relatively long and furthermore not temperature-controlled. The consequence thereof is temperature losses, pressure losses and dead volumes. The non-temperature-controlled connecting lines prove to be a very disruptive factor in particular when a plurality of reaction stages are to be performed at different temperature levels and accordingly a plurality of thermostats are used.

### SUMMARY OF INVENTION

The object underlying the invention is therefore to enable system-external fluidic components to be integrated into a microfluidic system having short connecting paths and good temperature controllability.

The object is achieved according to the invention by means of a module for a modular microfluidic system in which modules arranged adjacent to one another in a row are fluidically connected to one another by means of connecting parts containing connection channels, the module having the following features:
the module has a plate-shaped microfluidic part which contains a fluid channel system and on its top side in edge regions to the potentially adjacent modules of the microfluidic system has fluid terminals, wherein the fluidic connection to adjacent modules can be established by means of the connecting parts abutting in edge regions on the top side,
disposed below the microfluidic part is an insulation vessel which can be filled with a temperature control fluid and through which said temperature control fluid can flow and which is closed off at the top by the microfluidic part serving as a lid,
the microfluidic part has, on its underside, connecting means for fluidically connecting a fluidic component which can be housed in the insulation vessel to the fluid channel system of the microfluidic part, and
the microfluidic part and/or the insulation vessel have/has securing means for holding the fluidic component.

Thus, a separate module which is disposed, in common with all the other modules, in the microfluidic system is provided for the system-external fluidic component. The system-external fluidic component is in this case connected via short tubes or capillaries to the microfluidic part of the respective module and thereby integrated into the microfluidic system. Both the fluidic component and the tubes or capillaries for connecting to the microfluidic part and the microfluidic part itself with the fluid channel system contained therein are temperature-controlled, i.e. heated or cooled, by means of the temperature control fluid in the interior of the insulation vessel.

The temperature control fluid is preferably circulated in a temperature control fluid circuit so that the temperature control fluid flows continuously through the insulation vessel and the temperature of the temperature control fluid outside of the module can be regulated for example by means of a thermostat.

In order to be able to regulate or change the temperature control of the fluidic component quickly for example in the case of exothermic reactions or for terminating reactions, the temperature control fluid is preferably mixed from a hot fluid feed and a cold fluid feed by means of a controllable mixing device.

In an advantageous development of the module according to the invention, the insulation vessel has an inlet for the temperature control fluid in its lower region and, disposed on the underside of the plate-shaped microfluidic part, an outlet which leads into a separate temperature control fluid channel of the fluid channel system. In this way it is possible to control the temperature in the interior of the microfluidic part directly so that no temperature gradient is produced in the upper region of the insulation vessel and the fluidic component can be arranged very close to the microfluidic part in the interest of achieving short connecting paths. In addition this means that the temperature of the fluids can continue to be controlled after they exit the fluidic component. For that purpose the separate temperature control fluid channel runs inside the microfluidic part preferably in thermal contact with predefined fluid channels of the fluid channel system.

In order to be able also to control the temperature of the connecting parts or, as the case may be, the connection channels contained therein it can be provided that the separate temperature control fluid channel leads into at least one separate fluid terminal on the top side of the plate-shaped microfluidic part. The relevant connecting part contains an additional temperature control fluid channel for the purpose of connecting to the separate temperature control fluid channel of the microfluidic part, the additional temperature control fluid channel running inside the connecting part preferably in thermal contact with predefined connection channels.

The separate temperature control fluid channel in the microfluidic part leads, where appropriate via the additional temperature control fluid channel in the connecting part, preferably to an outlet terminal from which the temperature control fluid can be routed further in the temperature control fluid circuit. In order to keep non-temperature-controlled, uninsulated or subsequently to be insulated connecting lines in the temperature control fluid circuit as short as possible, the outlet terminal is routed via a temperature control fluid line through the insulation vessel to the lower region of the insulation vessel with the inlet disposed there, and at that point exits the insulation vessel.

### BRIEF DESCRIPTION OF THE DRAWINGS

In order to explain the invention in further detail, reference is made in the following to the figures of the drawing, in which:

FIG. 1 shows an exemplary embodiment of a modular microfluidic system,

FIG. 2 shows the upper part of one of the modules comprising a microfluidic part and connecting parts,

FIG. 3 shows an example of the plate-shaped microfluidic part,

FIG. 4 shows an example for installing the microfluidic parts in the modules and the fluidic connection of the microfluidic parts of two adjacent modules by means of the connecting part,

FIG. 5 shows the upper part of a module in a section along the module row, and

FIG. 6 shows an exemplary embodiment of the module according to the invention.

### DETAILED DESCRIPTION OF INVENTION

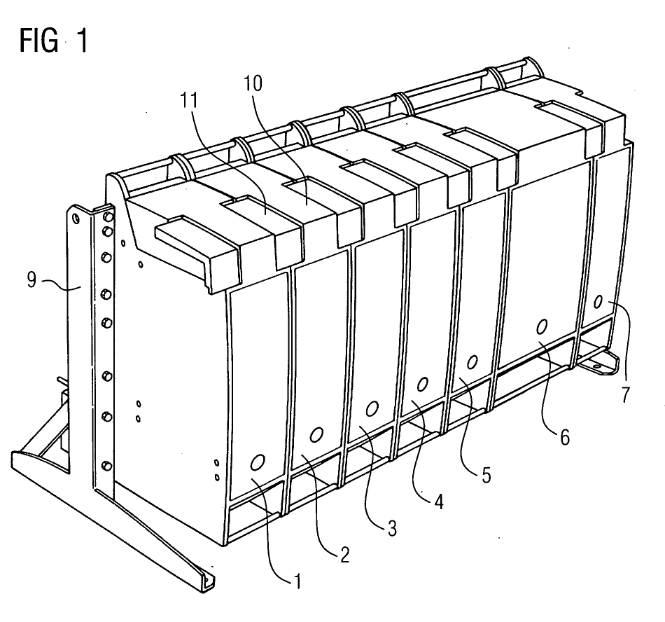

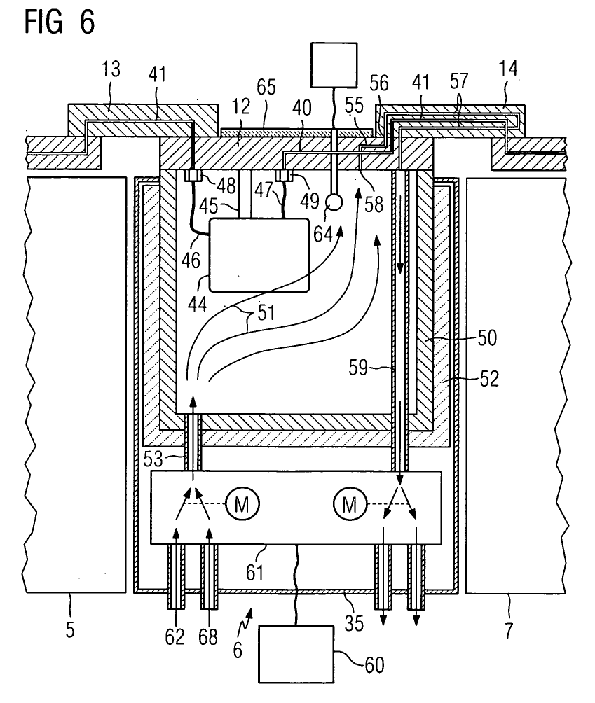

FIG. 1 shows a microfluidic system comprising modules 1 to 7 which are arranged adjacent to one another in a row and are held at the rear on a carrier frame 9. In this array the modules 1 and 7 form the end modules, i.e. the start and end modules, of the microfluidic system. Each module 1 to 7 contains a microfluidic part and an associated electrical control unit. The control units of the different modules are interconnected via an electrical line bus and the microfluidic parts via a fluid bus. The electrical line bus runs in the carrier frame 9, the modules 1 to 7 being removably connected to the line bus via rear-side plug-in connectors. The fluid bus is formed by connecting parts which contain connection channels and fluidically connect the microfluidic parts of respectively adjacent modules 1 to 7 to one another. The microfluidic parts are disposed in the region of the top sides of the modules and during normal operation of the microfluidic system are covered by protective covers 10 removably attached to the modules 1 to 7. The connecting parts connecting the microfluidic parts of respectively adjacent modules 1 to 7 are covered by further protective hoods 11. In the exemplary embodiment shown here, the module labeled by the reference sign 6 and having twice the width of the remaining modules 1, 2, 3, 4, 5 and 7 serves for housing and controlling the temperature of a system-external fluidic component. Said module 6 will be explained in more detail later with reference to FIG. 6.

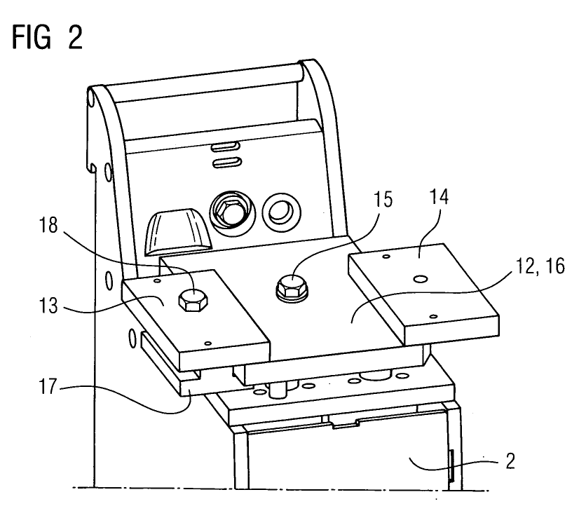

FIG. 2 shows the upper part of one of the modules, e.g. 2, with protective hoods 10, 11 removed so that the microfluidic part 12 and the connecting parts 13 and 14 to the adjacent modules 1 and 3 can be seen. The plate-shaped microfluidic part 12 is seated with its underside in a locally limited area of the plate center on a bearing surface of the module 2 and is pressed against said bearing surface by means of a releasable securing element 15 in the form of a bolt. The microfluidic part 12 contains a fluid channel system comprising fluid terminals which are arranged on the top side 16 of the microfluidic part 12 in the edge regions toward the microfluidic parts of the adjacent modules 1 and 3. The fluid terminals of each two adjacent microfluidic parts, e.g. 12 and the corresponding microfluidic part of the module 1, are connected to one another by means of the connection channels in the connecting part, e.g. 13, which, spanning the two microfluidic parts, rests on their top sides in the edge regions. In the opposing end regions on the undersides of the two adjacent microfluidic parts there rests a clamping part 17 which is connected in the area between the two microfluidic parts via a further releasable securing element 18, likewise in the form of a bolt, to the connecting part 13 and presses the latter against the top sides of the two microfluidic parts.

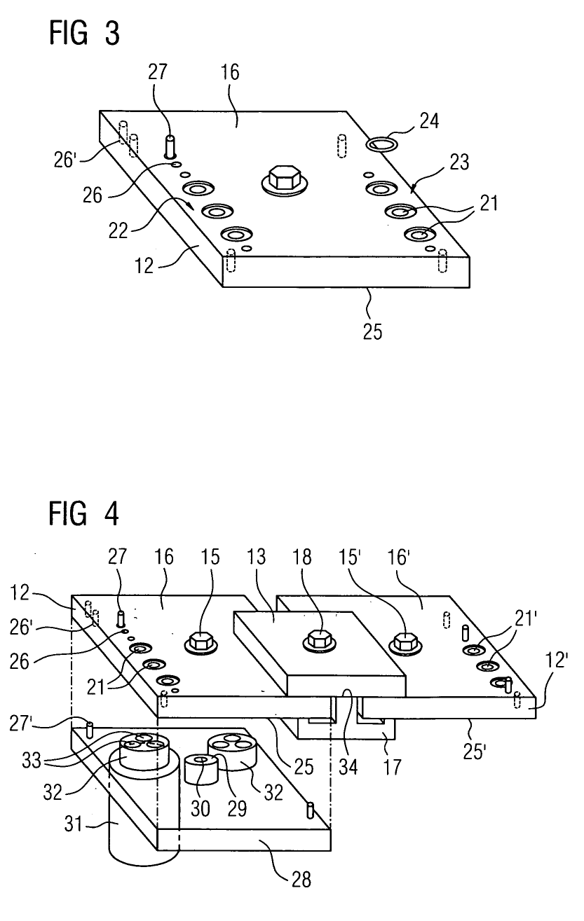

FIG. 3 shows an example of the plate-shaped microfluidic part 12, which can be embodied as a single plate or in the form of a plate composite made of steel, glass, silicon or another suitable material. Within the plate or plates, fluid channels of a fluid channel system run essentially parallel to the two large-area plate main sides and are connected at right angles thereto to the fluid terminals 21 in the edge regions 22 and 23 of the top side 16 of the microfluidic part 12. The fluid terminals 21 contain recesses for accommodating elastic sealing means 24 in the form of sealing washers. Provided on the top side 16 and the underside 25 of the microfluidic part 12 are positioning means in the form of drilled holes 26 and 26′ for receiving pilot pins 27 and 27′ which serve for aligning the microfluidic part 12 in relation to the module to be housed or for aligning the connecting parts in relation to the microfluidic part 12. In this case the positioning means 26, 26′, 27, 27′ are preferably embodied or arranged according to a predefined coding scheme which only allows predefined combinations of microfluidic part and module or, as the case may be, connecting part and microfluidic part.

FIG. 4 shows an adapter plate 28 which can be secured to the top side of the module and in the center of which there is embodied the bearing surface 29 for the microfluidic part 12. The bearing surface 29 contains an internal thread into which the bolt 15 can be screwed so that the microfluidic part 12 is pressed by means of the bolt 15 against the bearing surface 29 in the area of the plate center. The hole-pin combinations 26′, 27′ ensure that on the one hand only a microfluidic part 12 that is allowed for the relevant module can be mounted on the adapter plate 28 and that on the other hand the microfluidic part 12 is correctly aligned in its location. At least one further micro- and/or macrofluidic unit 31 can be installed on the underside of the adapter plate 28. In the example shown here, the microfluidic part 12 contains on its underside 25 additional fluid terminals which serve for connecting at least one further micro- or macrofluidic unit 31. Said further micro- or macrofluidic units 31 can be pumps, valves, measuring equipment or analytical instruments, etc. which because of their size or for other reasons are not integrated in the microfluidic units, but are otherwise essential components of the modules. The further micro- or macrofluidic units 31 are housed inside the module in a space under the adapter plate 28 and connected via fluid terminal adapters 32 to the additional fluid terminals on the underside 25 of the microfluidic part 12. The fluid terminal adapters 32 are arranged in an easily replaceable manner on the adapter plate 28 and have, on their top sides which project as far as the underside 25 of the microfluidic part 12, the fluid terminals 33 of the further micro- or macrofluidic units 31 for connecting to the microfluidic part 12. Different adapter plates 28 can be provided for different additional micro- and/or macrofluidic units 31.

FIG. 4 also shows once again the fluidic connection of the microfluidic parts 12 and 12′ of two adjacent modules by means of the connecting part 13, which spans the two microfluidic parts 12 and 12′ and at the same time bears on their top sides 16 and 16′ in the edge regions containing the fluid terminals 21, 21′ and disposed adjacent to one another. In the opposing edge regions on the undersides 25 and 25′ of the two microfluidic parts 12, 12′ there rests the clamping part 17 which is connected in the area between the two microfluidic parts 12, 12′ via the further bolt 18 to the connecting part 13 and presses the latter against the top sides 16 and 16′ of the two microfluidic parts 12 and 12′. In the area between the two microfluidic parts 12 and 12′ the clamping part 17 has a further bearing surface 34 for the connecting part 13, said bearing surface lying at least approximately in the plane of the top sides 16 and 16′ of the microfluidic parts 12 and 12′, such that in the installed state the connecting part 13 butts against said further bearing surface 34 and cannot be deflected further or broken under the pressure exerted by the bolt 18.

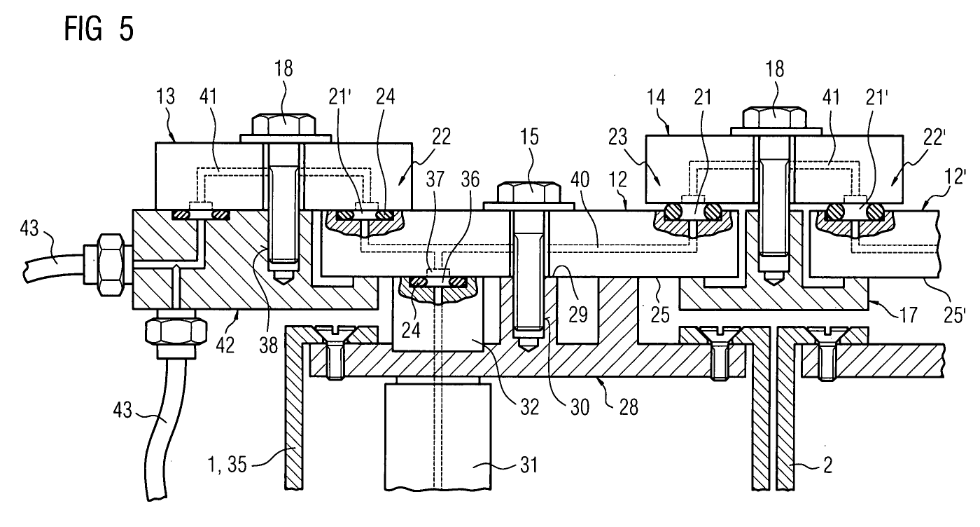

FIG. 5 shows the upper part of the end module 1 and part of the module 2 in a section longitudinally with respect to the module row. Installed in the upper region of the module housing 35 is the adapter plate 28 which on its top side carries a fluid terminal adapter 32 for a further micro- or macrofluidic unit 31. The unit 31 is installed in the housing 35 and fluidically connected from below to the fluid terminal adapter 32. The further fluid terminals 36 of the unit 31 for connecting to the microfluidic part 12 are formed on the top side of the fluid terminal adapter 32. The microfluidic part 12 butts with its underside 25 in the area of the plate center on the bearing surface 29 embodied for that purpose on the adapter plate 28 and containing the internal thread 30 for screwing in the bolt 15, such that the microfluidic part 12 is pressed by means of the bolt 15 against the bearing surface 29 in the area of the plate center. The adapter plate 20 also has an auxiliary bearing surface 39 for the microfluidic part 12 which is arranged symmetrically to the fluid terminal adapter 32 in relation to the plate center.

In its interior the microfluidic part 12 contains fluid channels 40 which, depending on the function of the module 1, form for example a reactor, a mixer or a dwell line for fluids or a plurality of functional units of said type, and run parallel to the top side and underside 16 and 25, respectively, of the planar microfluidic part 12. Those fluid channels 40 which are provided for connecting to fluid channels in the microfluidic parts of potentially adjacent modules, in this case e.g. the module 2, lead to the fluid terminals 21 which are contained on the top side 16 of the microfluidic part 12 in the edge regions 22 and 23 to the potential adjacent modules. Additional fluid terminals 37 on the underside 25 of the microfluidic part 12 serve for connecting the further micro- or macrofluidic unit 31.

The fluid terminals 21, 21′ of the adjacent microfluidic parts 12 and 12′ are connected to one another by means of the connection channels 41 in the connecting part 14 which spans the two microfluidic parts and at the same time bears on their top sides in the edge regions 23, 22′. Bearing in the same edge regions 23, 22′ against the undersides 25, 25′ of the two microfluidic parts 12 and 12′ is the clamping part 17 which is connected in the area between the two microfluidic parts 12 and 12′ via the further bolt 18 to the connecting part 14 and presses the latter against the top sides of the two microfluidic parts 12 and 12′. The connecting part 14 is likewise embodied as a plate or plate composite and is preferably formed from the same material as the microfluidic parts 12, 12′ so that the formation of electrical local elements is prevented.

The elastic sealing washers 24 disposed in recesses in the area of the fluid terminals 21, 21′ are compressed by the contact pressure of the connecting part 14 and seal off the fluid connections to the outside. At the same time the sealing washers 24 to a certain extent allow different thickness tolerances or orientation and location tolerances of the respectively adjacent microfluidic parts 12, 12′ in the vertical direction (height offset), without jeopardizing the leak tightness of the system.

As FIG. 5 also shows, a fluid terminal part 42 for connecting external fluid lines 43 is provided for the end module 1 in order to enable fluids to be supplied or drained off at the end module 1 of the microfluidic system. The fluid terminal part 42 is secured by means of the further bolt 18 to the underside of the connecting part 13 instead of a clamping part 17 and in the process connects the connection channels 41 in the connecting part 13 to the external fluid lines 43.

FIG. 6 shows the module 6 (cf. FIG. 1) which serves to accommodate a system-external fluidic component 44, e.g. a reactor. The fluidic component 44 is held with the aid of securing means 45 to the underside of the microfluidic part 12 at a distance from the latter and connected via tubes 46, 47 to connecting means 48, 49 on the underside of the microfluidic part 12 via which it is fluidically connected to predefined fluid channels 40 in the microfluidic part 12. The fluid channels 40 are in turn fluidically connected via the connection channels 41 in the connecting parts 13, 14 to the adjacent modules 5 and 6. The fluidic component 44 is located in an insulation vessel 50 which is closed off at the top in the manner of a lid by means of the microfluidic part 12 and is completely filled with a temperature control fluid 51 and through which said temperature control fluid 51 flows. The insulation vessel 50 can be embodied as a Dewar vessel and is in this case provided with an outer insulation 52. The microfluidic part 12 carries a heat insulation 65 on its top side. The temperature control fluid 51 is circulated in a temperature control fluid circuit and arrives in the insulation vessel 50 via an inlet 53 in the lower region of the vessel 50. The temperature control fluid 51 exits the vessel 50 via an outlet 54 on the underside of the microfluidic part 12 which leads into a separate temperature control fluid channel 55 of the fluid channel system of the microfluidic part 12. The separate temperature control fluid channel 55 runs inside the microfluidic part 12 in thermal contact with the fluid channel 40 which conveys the reactant coming from the reactor 44, and leads to a separate fluid terminal 56 on the top side of the microfluidic part 12. From there the temperature control fluid 51 is routed in an additional temperature control fluid channel 57 of the connecting part 14 in thermal contact with the connection channels 41 disposed there and finally back once more into the microfluidic part 12. The temperature control fluid 51 exits the microfluidic part 12 on the latter's underside via an outlet terminal 58 and from there is routed via a temperature control fluid line 59 through the insulation vessel 50 to the lower region of the vessel 50 where it exits the latter. In order to empty the insulation vessel 50 the tubular temperature control fluid line 59 can be removed, e.g. unscrewed.

The module 6 also contains a mixing device 61 that is controllable by means of a control device 60 for the purpose of mixing the temperature control fluid 51 from a hot fluid feed 62 and a cold fluid feed 63. A temperature sensor 64 which can be connected to the control device 60 is installed in the upper region of the insulation vessel 50 above the microfluidic part 12. Baffle parts (not shown here) can be arranged in the insulation vessel 50 in order to improve the heat transfer at the fluidic component.

1.-10. (canceled)

11. A module for a modular microfluidic system in which modules arranged adjacent to one another in a row are fluidically interconnected by means via connecting parts containing connection channels, comprising:
a plate-shaped microfluidic part which contains a fluid channel system and on a top side in edge regions to the potentially adjacent modules of the microfluidic system has fluid terminals, wherein the fluidic connection to adjacent modules is established via the connecting parts abutting in edge regions on the top side;
an insulation vessel arranged below the microfluidic part filled with a temperature control fluid and through which the temperature control fluid flows and which is closed off at the top by the microfluidic part that serves as a lid to the insulation vessel;
a connecting device that fluidically connects a fluidic component, the connecting device being arranged on an underside of the microfluidic part and housed in the insulation vessel to the fluid channel system of the microfluidic part; and
a securing device that holds the fluidic component, the securing device being associated with microfluidic part or the insulation vessel.

12. The module as claimed in claim 11, wherein the temperature control fluid is circulated in a temperature control fluid circuit.

13. The module as claimed in claim 12, further comprising a controllable mixing device that mixes a hot fluid feed and a cold fluid feed to form the temperature control fluid.

14. The module as claimed in claim 13, wherein the insulation vessel has, in a lower region, an inlet for the temperature control fluid and, on the underside of the plate-shaped microfluidic part, an outlet that leads to a separate temperature control fluid channel of the fluid channel system.

15. The module as claimed in claim 14, wherein the separate temperature control fluid channel runs inside the microfluidic part in thermal contact with predefined fluid channels of the fluid channel system.

16. The module as claimed in claim 15, wherein the separate temperature control fluid channel leads into at least one separate fluid terminal on the top side of the plate-shaped microfluidic part.

17. The module as claimed in claim 16, wherein at least one of the connecting parts contains an additional temperature control fluid channel that connects to the separate temperature control fluid channel of the microfluidic part.

18. The module as claimed in claim 17, wherein the additional temperature control fluid channel runs inside the connecting part in thermal contact with predefined connection channels.

19. The module as claimed in claim 18, wherein the separate temperature control fluid channel leads, where appropriate via the additional temperature control fluid channel, to an outlet terminal in the microfluidic part, from which outlet terminal the temperature control fluid is routed further in the temperature control fluid circuit.

20. The module as claimed in claim 19, wherein the outlet terminal is routed via a temperature control fluid line through the insulation vessel to the lower region of the insulation vessel where the inlet to the temperature control fluid line is arranged and the at that point exits the insulation vessel.
